(19) United States
(12) Patent Application Publication
(10) Pub. No.: US 2005/0042622 A1
(43) Pub. Date: Feb. 24, 2005

(54) Computer based method and program for evaluating candidate genes

(75) Inventor: Janet A. Warrington, Los Altos, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/687,480

(22) Filed: Oct. 15, 2003

Related U.S. Application Data
(60) Provisional application No. 60/418,680, filed on Oct. 15, 2002.

(51) Int. Cl. C12Q 1/68; G06F 19/00; G01N 33/48; G01N 33/50

(52) U.S. Cl. 435/6; 702/20

(57) ABSTRACT

A computer based method and computer program product are presented for evaluating information concerning candidate genes and experiments used to identify the candidate genes, wherein the candidate genes are identified via examining expression levels of a plurality of genes, wherein the expression levels are measured by conducting hybridization experiments with nucleic acid microarray chips.

The present application claims priority to U.S. Provisional Patent Application ser. No. 60/418,680, titled “Computer Based Method and Program for Evaluating Candidate Genes”, filed Oct. 15, 2002, which is hereby incorporated by reference herein in its entirety for all purposes.

### FIELD OF THE INVENTION

This invention relates in general to methods for evaluating candidate genes Identified by experiments using nucleic acid arrays and in particular to computer based methods for evaluating candidate genes.

### BACKGROUND OF THE INVENTION

New technology has enabled the production of microarrays smaller than a thumbnail that contain hundreds of thousands or more of different molecular probes. These techniques are described in U.S. Pat. No. 5,143,854, PCT WO 92/10092, and PCT WO 90/15070. Microarrays have probes arranged in arrays, each probe ensemble assigned a specific location. Microarrays have been produced in which each location has a scale of, for example, ten microns. The microarrays can be used to determine whether target molecules interact with any of the probes on the microarrays. After exposing the array to target molecules under selected test conditions, scanning devices can examine each location in the array and determine whether a target molecule has interacted with the probe at that location.

Microarrays wherein the probes are oligonucleotides (“oligonucleotide arrays”) show particular promise. Arrays of nucleic acid probes can be used to extract sequence information from nucleic acid samples. The samples are exposed to the probes under conditions that allow hybridization. The arrays are then scanned to determine to which probes the sample molecules have hybridized. One can obtain sequence information by selective tiling of the probes with particular sequences on the arrays, and using algorithms to compare patterns of hybridization and non-hybridization. This method is useful for sequencing nucleic acids. It is also useful in gene expression monitoring, i.e., monitoring the expression of a multiplicity of preselected genes.

There is a need for methods for evaluating candidate genes identified by nucleic acid arrays and in particular for genes identified by oligonucleotide arrays. More particularly, there is a need for computer based methods for evaluating candidate genes.

### SUMMARY OF THE INVENTION

A computer based method and computer program product are presented for evaluating information concerning candidate genes and experiments used to identify the candidate genes, wherein the candidate genes are identified via examining expression levels of a plurality of genes, wherein the expression levels are measured by conducting hybridization experiments with nucleic acid microarray chips. According to the instantly claimed method, pluralities of attributes concerning the experiment and candidate genes are collected, at least one plurality of groupings is defined; based upon the groupings, information is selected about the plurality of attributes to be evaluated; a plurality of resulting information is formed; and the plurality of resulting information is formatted for viewing by a user.

In the instantly claimed computer program product, code is provided collecting pluralities of attributes concerning the experiment and candidate genes, code is provided for defining at least one plurality of groupings; based upon the groupings, code is provided for selecting information about the plurality of attributes to be evaluated; code is provided for forming a plurality of resulting information; and code is provided for formatting the plurality of resulting for viewing by a user.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from I to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells. A Laboratory Manual, PCR Primer: A Laboratory Manual, and Molecular Cloning. A Laboratory Manual (all from Cold Spring Harbor Laboratory Press), Stryer, L. (1995) Biochemistry (4th Ed.) Freeman, New York, Gait, “Oligonucleotide Synthesis: A Practical Approach ” 1984, JRL Press, London, Nelson and Cox (2000), Lehninger, Principles of Biochemistry 3rd Ed., W.H. Freeman Pub., New York, N.Y. and Berg et al. (2002) Biochemistry, 5th Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1,17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. No. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80:1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent applications Ser. Nos. 10/063,559, 60/349,546, 60/376,003, 60/394,574, 60/403,381.

I. Evaluating Candidate Genes

According to the present invention, candidate genes may be evaluated by considering some or all of the following questions and information:
1. How sample quality was evaluated.
2. How RNA integrity was evaluated.
3. What specifications were used for acceptance of a sample into the study?
4. What was the goal of the study?
5. What medical question was being addressed?
6. What samples were used, how and why were they selected?
7. Is there any previous work/data supporting the selection of these samples as a model for addressing this question?
8. What are the known weaknesses of this model system?
9. What controls were used as baseline or normal?
10. Were the samples matched?
11. How many samples were ultimately used in the analysis?
12. How was the data filtered?
13. How were false positives managed/eliminated/minimized? False negatives?
14. What statistical methods were employed for the analysis? How was statistical significance determined? What thresholds were used?
15. What is the range of differences in expression level observed for the candidates? What is the median? The mode?
16. Were candidates genes validated by other methods? Which methods? How many candidates were evaluated?
17. How many outlier patients were identified?
18. How was ambiguous information processed?
19. Was clinical information integrated into the analysis? How many categories? What categories?
20. Were candidates selected based on differences seen in every patient or in a number of patients but not all patients? Were any candidates outliers in a subset of patients? How many outlier genes were identified? Were they eliminated from the candidate set?
21. Were candidates functionally characterized? Were previously known markers identified? New relationships with known pathways?

According to the present invention, a computer based method is presented for evaluating information concerning candidate genes and experiments used to identify the candidate genes, wherein the candidate genes are identified via examining expression levels of a plurality of genes, wherein the expression levels are measured by conducting hybridization experiments with nucleic acid microarray chips. The instantly claimed method has the following steps:
collecting a plurality of sample attributes from the experiments;
collecting a plurality of study attributes from the experiments;
collecting a plurality of control attributes from the experiments;
collecting a plurality of data attributes from the experiments;
collecting a plurality of false positive/negative attributes from the experiments;
collecting a plurality of literature attributes concerning the candidate genes;
collecting a plurality of patient attributes concerning the candidate genes;
collecting a plurality of clinical information attributes concerning the candidate genes;
collecting a plurality of validation attributes concerning the candidate genes;
collecting a plurality of functional attributes concerning the candidate genes;
defining at least one of a plurality of groupings of the attributes;
selecting, based upon at least one of a plurality of groupings, information about the plurality of attributes to be evaluated;
forming a plurality of resulting information;
and formatting the plurality of resulting information for viewing by a user.

According to the present invention, it is preferred that the plurality of sample attributes is selected from the group consisting of sample quality data, sample matching information, total sample number information, and sample selection criterion. It is also preferred that the plurality of study attributes is selected from the group consisting of the goal of the study, medical question addressed by the study, and known weaknesses of any model system employed in the study. It is also preferred that the plurality of control attributes is selected from the group consisting of normalizing controls and baseline controls. It is also preferred that the plurality of data attributes is selected from the group consisting of data filtration information, statistical methods employed in the analysis, including how statistical significance was determined and what thresholds were used, and range of expression level observed for the candidate genes. It is also preferred that the plurality of false positive/negative attributes is selected from the group consisting of information on false positive management and information on false negative management. It is also preferred that the nucleic acid microarray chip is an oligonucleotide microarray chip.

In another aspect of the present invention, a computer program product is presented for evaluating information concerning candidate genes and experiments used to identify the candidate genes, wherein the candidate genes are identified via examining expression levels of a plurality of genes, wherein the expression levels are measured by conducting hybridization experiments with nucleic acid microarray chips. The instantly claimed computer program product has the following components:
code for collecting a plurality of sample attributes from the experiments;
code for collecting a plurality of study attributes from the experiments;
code for collecting a plurality of control attributes from the experiments;
code for collecting a plurality of data attributes from the experiments;
code for collecting a plurality of false positive/negative attributes from the experiments;
code for collecting a plurality of literature attributes concerning the candidate genes;
code for collecting a plurality of patient attributes concerning the candidate genes;
code for collecting a plurality of clinical information attributes concerning the candidate genes;
code for collecting a plurality of validation attributes concerning the candidate genes;
code for collecting a plurality of functional attributes concerning the candidate genes;
code for defining at least one of a plurality of groupings of the attributes;
code for selecting, based upon the at least one of a plurality of groupings,
information about the plurality of attributes to be evaluated;
code for forming a plurality of resulting information;
and code for formatting the plurality of resulting information for viewing by a user.

According to the instant invention, preferred embodiments of the instantly claimed computer program product are as set forth with respect to the computer based method.

The foregoing invention has been described in some detail by way of illustration and examples, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1) A computer based method for evaluating information concerning candidate genes and experiments used to identify said candidate genes, wherein said candidate genes are identified via examining expression levels of a plurality of genes, wherein said expression levels are measured by conducting hybridization experiments with nucleic acid microarray chips, said method comprising:
collecting a plurality of sample attributes from said experiments;
collecting a plurality of study attributes from said experiments;
collecting a plurality of control attributes from said experiments;
collecting a plurality of data attributes from said experiments;
collecting a plurality of false positive/negative attributes from said experiments;
collecting a plurality of literature attributes concerning said candidate genes;
collecting a plurality of patient attributes concerning said candidate genes;
collecting a plurality of clinical information attributes concerning said candidate genes;
collecting a plurality of validation attributes concerning said candidate genes;
collecting a plurality of functional attributes concerning said candidate genes;
defining at least one of a plurality of groupings of said attributes;
selecting, based upon said at least one of a plurality of groupings, information about said plurality of attributes to be evaluated;
forming a plurality of resulting information;
and formatting said plurality of resulting information for viewing by a user.

2) A computer based method according to claim 1 wherein said plurality of sample attributes are selected from the group consisting of sample quality data, sample matching information, total sample number information, and sample selection criterion.

3) A computer based method according to claim 1 wherein said plurality of study attributes are selected from the group consisting of the goal of the study, medical question addressed by the study, and known weaknesses of any model system employed in the study.

4) A computer based method according to claim 1 wherein said plurality of control attributes is selected from the group consisting of normalizing controls and baseline controls.

5) A computer based method according to claim 1 wherein said plurality of data attributes is selected from the group consisting of data filtration information, statistical methods employed in the analysis, including how statistical significance was determined and what thresholds were used, and range of expression level observed for the candidate genes.

6) A computer based method according to claim 1 wherein said plurality of false positive/negative attributes is selected from the group consisting of information on false positive management and information on false negative management.

7) A computer based method according to claim 1 wherein said nucleic acid microarray chip is an oligonucleotide microarray chip.

8) A computer program product for evaluating information concerning candidate genes and experiments used to identify said candidate genes, wherein said candidate genes are identified via examining expression levels of a plurality of genes, wherein said expression levels are measured by conducting hybridization experiments with nucleic acid microarray chips, said computer program product comprising:
code for collecting a plurality of sample attributes from said experiments;
code for collecting a plurality of study attributes from said experiments;
code for collecting a plurality of control attributes from said experiments;
code for collecting a plurality of data attributes from said experiments;
code for collecting a plurality of false positive/negative attributes from said experiments;
code for collecting a plurality of literature attributes concerning said candidate genes;
code for collecting a plurality of patient attributes concerning said candidate genes;
code for collecting a plurality of clinical information attributes concerning said candidate genes;
code for collecting a plurality of validation attributes concerning said candidate genes;
code for collecting a plurality of functional attributes concerning said candidate genes;
code for defining at least one of a plurality of groupings of said attributes;
code for selecting, based upon said at least one of a plurality of groupings, information about said plurality of attributes to be evaluated;
code for forming a plurality of resulting information;
and code for formatting said plurality of resulting information for viewing by a user.

9) The computer program product of claim 8 wherein said plurality of sample attributes is selected from the group consisting of sample quality data, sample matching information, total sample number information, and sample selection criterion.

10) The computer program product of claim 8 wherein said plurality of study attributions is selected from the group consisting of the goal of the study, medical question addressed by the study, and known weaknesses of any model system employed in the study.

11) The computer program product of claim 8 wherein said plurality of control attributes is selected from the group consisting of normalizing controls and baseline controls.

12) The computer program product of claim 8 wherein said plurality of data attributes is selected from the group consisting of data filtration information, statistical methods employed in the analysis, including how statistical significance was determined and what thresholds were used, and range of expression level observed for the candidate genes.

13) The computer program product of claim 8 wherein said plurality of false positive/negative attributes is selected from the group consisting of information on false positive management and information on false negative management.

14) The computer program product of claim 8 wherein said nucleic acid microarray chip is an oligonucleotide microarray chip.
